(19) United States
(12) Patent Application Publication
(10) Pub. No.: US 2023/0072515 A1
(43) Pub. Date: Mar. 9, 2023

(54) A COLD THERAPY DEVICE AND SYSTEM

(71) Applicant: THE PROPERTY OF DSC LTD, Swanage Dorset (GB)

(72) Inventor: Dean Skivington, Swanage Dorset (GB)

(21) Appl. No.: 17/983,586

(22) Filed: Nov. 9, 2022

Related U.S. Application Data
(63) Continuation of application No. PCT/GB2021/051159, filed on May 13, 2021.

(30) Foreign Application Priority Data
May 13, 2020 (GB) 2007071.0

(51) Int. Cl. A61F 7/00

(52) U.S. Cl. CPC A61F 7/0053; A61F 7/0085; A61F 2007/0063; A61F 2007/0075; A61F 2007/0094

(57) ABSTRACT

A cold therapy device for use in a water delivery system which is coupled to a mains water supply, the device includes a housing having an inlet for receiving water from a mains water supply, the housing includes at least one cooling module configured to reduce the temperature within the housing to cold therapeutic temperatures, so that, in use, when water is received therein from the inlet, it is cooled to cold therapeutic temperatures. The device further includes an outlet for delivering water from the housing to the user.

### CROSS-REFERENCE TO RELATED APPLICATIONS

This application is a continuation of PCT International Application PCT/GB2021/051159, filed on May 13, 2021, which claims the benefit of, or priority to, United Kingdom Patent Application Serial Number 2007071.0, filed on May 13, 2020, both of which are hereby incorporated by reference in their entirety for all purposes.

### BACKGROUND OF THE INVENTION

Cold therapy (also referred to as cryotherapy) is a term used to describe the use of super-cool, freezing, and near-freezing temperatures to treat lesions, muscular aches and pain, and inflammation, to improve circulation and to aid weight loss, as well as other applications. For athletes, cold therapy can be an important part of their training routine, providing pain relief and promoting muscular healing. Many cold therapy techniques exist, and one such technique is to use supercool fluids, such as liquid nitrogen, in a cryotherapy chamber to bring the ambient temperature within the chamber to as low as around minus 110° C. or less to provide partial body cryotherapy (PBC) or whole-body cryotherapy (WBC). In PBC, a subject might place part of their body to be treated, for example an arm or leg, into a cryotherapy chamber. In WBC, the subject is entirely exposed to the low temperature by placing their entire body in the cryotherapy chamber. WBC is often used by athletes who require whole body therapy and for weight loss applications and improving circulation.

Cryotherapy chambers and equipment suitable for administering cryotherapy techniques tend only to be available through specialist clinics or centres, or through specially trained therapists. Home installation of a cryotherapy chamber is, for many people, prohibitively expensive, and they require large amounts of power to run.

They also require a supply of liquid nitrogen which is difficult to obtain and store, adds still further to the expense. Further, the application of cold therapy techniques at a gym, hotels, offices and hospitals is not provided widely due to the high cost of providing this service.

Home-based cold therapy techniques can be performed using an ice bath, but may be inconvenient and is often inadequate because the ice bath may quickly warm to a temperature above the optimum temperature for therapeutic effect.

The present invention aims to address at least some of these issues.

### BRIEF SUMMARY OF THE INVENTION

In accordance with a first aspect of the present invention there is provided a cold therapy device for use in a water delivery system which is coupled to a mains water supply, the device comprising a housing having an inlet for receiving water from a mains water supply, the housing including at least one cooling module configured to reduce the temperature within the housing to cold therapeutic temperatures, so that, in use, when water is received therein from the inlet, it is cooled to cold therapeutic temperatures, the device further includes an outlet for delivering water from the housing on to the user.

A temperature for administering cold therapy may be any temperature in the continuous range of 0.5° C. to 5.5° C. The actual temperature will depend on the ambient temperature of the local water table. The present invention is configured to reduce the cold water temperature coming from the local water table to a temperature suitable for administering cold therapy.

In one exemplary embodiment of the invention the device may be shaped as a shower head attachment. Optionally, the outlet is a plate having a plurality of holes therethrough, so that water is delivered as a shower.

In an embodiment, the cooling module may include an enclosure having at least one cooling member therein and means for receiving a coolant fluid within a region adjacent said cooling member. Optionally, the enclosure may include an inlet for receiving the coolant fluid. The device may include means for selectively cooling the coolant fluid and delivering cooled coolant fluid to the enclosure. The coolant fluid may be glycol.

In one exemplary embodiment of the invention, the inlet may be configured to be removably attached to a shower inlet hose, and the outlet may be configured to be removably attached to a shower outlet hose.

In an embodiment of the invention the housing may include a chamber having a removable insert therein and optionally defining a plurality of channels for fluid flow. The removable insert may be filled with a coolant fluid. The coolant fluid may be glycol. The number of channels provide at least 60 m2 of surface are to contact water flowing therethrough. The number of channels can be varied according to the temperature of the local water table. The insert may be a passive cooling module. In one embodiment of the invention the at least one cooling module may be a Peltier module. Optionally, the device may include a thermally insulated chamber within the housing having an inlet in fluid communication with the housing inlet and an outlet in fluid communication with the housing outlet, wherein the at least one cooling module is optionally adjacent to the chamber and configured to reduce the temperature within the chamber.

In an exemplary embodiment of the invention the chamber may include tubing defining a flow path from the chamber inlet to the chamber outlet. Optionally the tubing is helical. This creates a longer flow path for the water to be cooled in, in use.

In accordance with an embodiment of the invention, the at least one cooling module may include a heat sink. Optionally, the housing may include a cavity in thermal contact with the heat sink of the at least one cooling module. In an embodiment, the cavity may include an inlet for receiving water from a domestic water supply and an outlet for delivering water received in the cavity, so that, in use, thermal energy is drawn away from the heat sink.

In an embodiment of the invention, the device may further include a selectively actuatable power source for delivering electrical power to the at least one cooling module. Optionally, the power source is a domestic power source. A domestic power source may be mains electricity.

In an embodiment the device may be configured to cool water to between 0.5° C. and 5.5° C. In some exemplary embodiments the device may be configured to cool water to below 10° C., and optionally configured to cool water to between 0.5° C. and 7.5° C.

In an exemplary embodiment the device may be configured to cool input water to the device by at least 5° C. Optionally, the device may be configured to cool input water to the device by at least 10° C.

A cold therapy kit for use in a water delivery system which is connected to a water supply, including a cold therapy device as described above including attachments for fitting the cold therapy device to a water delivery unit, the water delivery unit being configured to deliver water to the cold therapy device for cooling, and input means for selectively controlling the at least one cooling module.

In an embodiment of the invention, the at least one cooling module may be a Peltier module. Alternatively, the cooling module may be a passive cooling module, as described above. A passive cooling module may be an insert which is first cooled and then put into the cooling chamber. The insert may have a plurality of channels to allow water to flow therethrough.

In an embodiment of the invention the input means may be a remote control module configured to communicate with the at least one cooling module for remote selective control of the at least one cooling module. Optionally, the remote control module may be a program installed on a mobile device. For example, the program may be a mobile application.

In one embodiment of the invention, the water delivery system may be a shower, and the water delivery unit may be a shower unit. In an optional embodiment of the invention, the water delivery system may comprise waste water conduit configured to be connected to the input of a boiler, in use.

In an exemplary embodiment of the invention, the attachment means may be removable attachments, so that the cold therapy device can be removably attached to a water delivery unit.

Optionally, the cooling module may a chamber having a removable insert therein defining a plurality of channels for fluid flow. The removable insert may be filled with a coolant fluid. The coolant fluid may be glycol.

In accordance with a third aspect of the present invention there is provided a method for cooling water to cold therapy temperatures using a cold therapy device according to embodiments of the invention, wherein the cooling module includes a chamber having a removable insert therein defining a plurality of channels for fluid flow, the method including the steps of cooling the removable insert by placing it in a home refrigeration device;
inserting the cooled removable insert into the chamber of the cold therapy device
receiving cold water at the inlet from a domestic water supply
cooling the water to cold therapy temperatures within the cooling module by allowing the water to flow through the channels of the removable insert, and
delivering cooled water at the output for application to a user's body in cold therapy techniques.

The method supplies a user with an at home means of obtaining very cold water for application in cold therapy techniques, where the cooling module can be passively cooled without electrical cooling modules present. This is particularly desirable for travelling.

In accordance with a fourth aspect of the invention there is provided a method for cooling water to cold therapy temperatures using a cold therapy device according to embodiments of the invention, wherein the device is shaped as a shower head attachment, the method including the steps of;
attaching the inlet to a shower hose,
receiving cold water at the inlet from a domestic water supply,
cooling the cold water to cold therapy temperatures via the cooling module, and
delivering water at the outlet directly for application to a user's body in cold therapy techniques.

Optionally, the method may further include the step of splitting the water flow at the inlet and cycling the received cold water around the cooling module to aid cooling. This way water can be used to speed up cooling so that a lower temperature is achieved at the outlet, without slowing the speed of water flow.

In accordance with a further aspect of the invention there is provided a method of adapting a water delivery system to deliver water cooled to cold therapeutic temperatures, using a cold therapy device having an inlet for receiving water and an outlet for delivering cooled water, and having a cooling module therebetween, the method including the steps of
identifying the fluid flow path between the water source and the point of delivery of water;
making a discontinuation in the fluid flow path to create a first flow path coming from the domestic water source to the discontinuation and a second flow path going from the discontinuation to the point of delivery of water;
attaching the inlet of a cold therapy device to the first flow path at the discontinuation; and
attaching the outlet of a cold therapy device to the second flow path at the discontinuation, so that the fluid flow path is restored and water flows through the cooling module of the cold therapy device.

In an exemplary embodiment the domestic water source may be a boiler and the point of delivery of water is a shower. Optionally, the cooling module may include at least one Peltier module.

### BRIEF DESCRIPTION OF THE DRAWINGS

FIG. 1 is a schematic diagram of a cold therapy device according to a first embodiment of the present invention, suitable for use as a shower head;

FIG. 2A is a schematic diagram of a cold therapy device according to a second embodiment of the present invention, also suitable for use as a shower head;

FIG. 2B is a schematic diagram of the chamber of the cold therapy device of FIG. 2A;

FIG. 2C is a cross-sectional diagram across the line A-A of the chamber in the cold therapy device of FIG. 2A;

FIG. 3A is a schematic diagram of a cold therapy device according to a third exemplary embodiment of the invention;

FIG. 3B is a cross-sectional view of the handle of the cold therapy device of FIG. 3A.

FIG. 4 is a cold therapy system according to a fourth embodiment of the invention using the cold therapy device of FIG. 1, 2 or 3;

FIG. 5 is a schematic diagram of a cold therapy system according to a fifth embodiment of the invention using the cold therapy device of FIG. 1, 2 or 3;

FIG. 6A is a cross-sectional view diagram of a cold therapy device according to a sixth embodiment of the present invention, suitable for attaching, detaching and re-attaching to any existing shower system;

FIG. 6B is a cross-sectional side view of a cold therapy device according to an alternative sixth embodiment of the present invention;

FIG. 6C is a cross-sectional plan view of the cold therapy device of FIG. 6B;

FIG. 7 is a schematic diagram of a cold therapy system according to a seventh embodiment of the invention using the cold therapy device of FIGS. 6A or 6B and 6C;

FIG. 8 is a schematic diagram of a cold therapy system according to an eighth embodiment of the invention using the cold therapy device of FIG. 6;

FIG. 9 is a schematic diagram of a shower having a cold therapy system according to a ninth embodiment of the invention using the cold therapy device of FIG. 6;

FIG. 10 is a schematic diagram of a cold therapy device according to a tenth exemplary embodiment of the invention, suitable for use anywhere;

FIG. 11 is a schematic diagram of a cold therapy device according to an eleventh exemplary embodiment of the invention, suitable for use anywhere;

FIG. 12 is a schematic diagram of a cold therapy system according to an twelfth exemplary embodiment of the invention;

FIG. 13 is a schematic drawing of an alternative cooling chamber for use in any of the above described cold therapy devices or cold therapy systems of FIGS. 1 to 12;

FIG. 14A is a cross-sectional side view of a cold therapy device according to yet another exemplary embodiment of the invention;

FIG. 14B is a cross-sectional plan view of the cold therapy device of FIG. 14A;

FIG. 15 is a schematic view of the cold water outlet of the cold therapy device of FIG. 14;

FIG. 16 is a schematic perspective view of a cold therapy unit incorporating a c device according to the present invention;

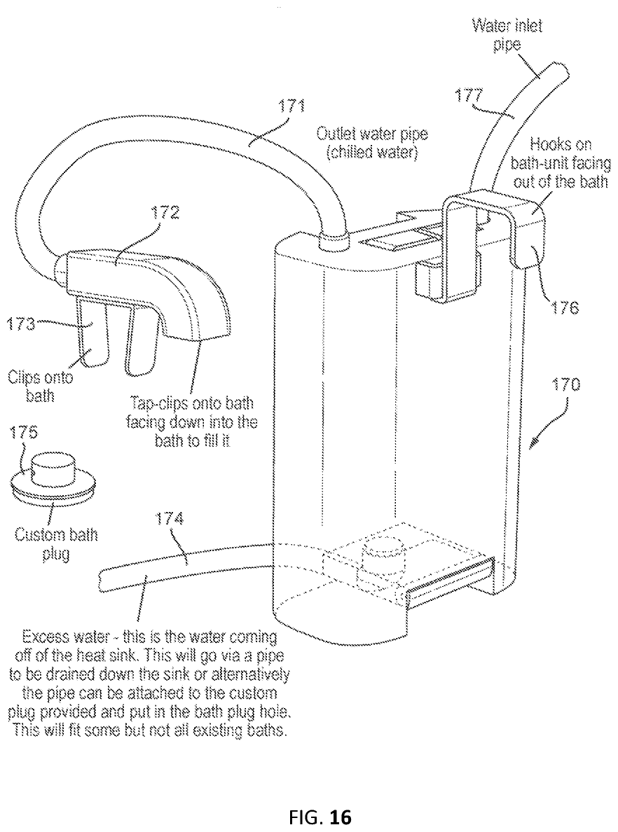

FIG. 17 is a schematic front view of a cold therapy device according to yet another exemplary embodiment of the invention;

FIG. 18 is a cross-sectional side view of one chamber of the cold therapy device of FIG. 17; and

FIG. 19 is a cross-sectional plan view of the cold therapy device of FIG. 17.

### DETAILED DESCRIPTION OF THE INVENTION

Directional descriptors such as upper, lower, left, right, clockwise, anti clockwise, front, rear and other similar adjectives are used for clarity and refer to the orientation of the invention as illustrated in the drawings, however it will be clear to those skilled in the art that the invention may not always be oriented as illustrated and the invention is not intended to be limited in this regard.

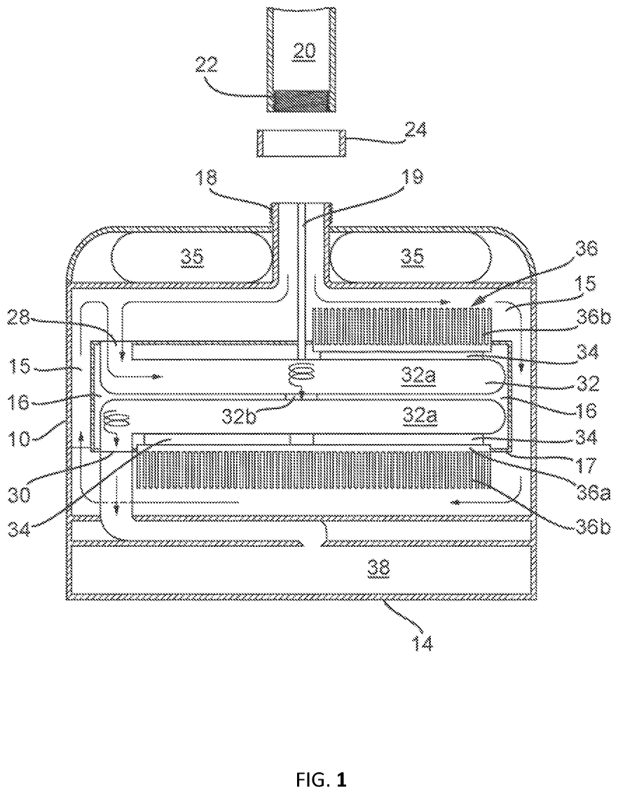

Referring to FIG. 1 of the drawings, a cold therapy device according to a first exemplary embodiment of the invention includes a housing 10 having an inlet 12 and an outlet 14. The housing defines a cavity 15 within it, accessible only by the inlet 12. The inlet 12 is a circular opening which protrudes from the upper surface of the housing 10. Within the cavity 15 is a chamber 16 having thermally insulated walls 17. The opening 12 includes a separator 19 in the form of a plate which splits the opening in two and extends from the opening to the upper insulated wall 17 of the chamber 16. The chamber 16 has a chamber inlet 28 and a chamber outlet 30, and the chamber inlet 28 is accessible via the cavity 15, while the chamber outlet 30 is connected to the housing outlet 14.

The outlet 14 to the housing 10 is constructed from a plurality of small openings, for example 2 mm in diameter, across the whole of the lower surface of the housing 10. The outlet 14 is accessible only via the chamber outlet 30. The outlet 14 is similar in design to any known shower head outlet, comprising a plurality of small openings to provide split a single stream of water into a plurality of streams of water, in use. For example, the outlet 14 may be a rainfall shower head outlet.

Additionally, the outlet 14 may include a misting device configured to diffuse water droplets in such a manner so as to deliver a fine mist to the user, in use.

An attachment means 18 is provided on the inlet 12 for enabling secure attachment to the end of a domestic water supply delivery device, for example, a shower hose 20. In this exemplary embodiment, the shower hose 20 comprises a screw threaded portion 22 and the attachment means 18 is a complementary screw threaded portion configured to engage with the screw threaded portion 22. A collar 24 is further provided to ensure a sealed connection between the housing and the shower hose 20. The shower hose 20 is still selectively controllable by a user by means of a faucet or shower unit.

The chamber 16 is thermally insulated from the cavity 15 by the insulating walls 17. This enables the inside of the chamber 16 to be kept at a lower temperature than the ambient temperature of the cavity 15. Suitable walls 17 for thermally insulating the chamber 16 are known in the art and the present invention is not necessarily intended to be limited in this regard. For example, the chamber walls 17 may be formed from two layers having a cavity therebetween. The cavity may include an insulating material such as extruded polystyrene foam, an air-gap, or a vacuum. The chamber walls 17 may be constructed from a material having a low thermal conductivity.

The chamber inlet 28 and chamber outlet 30 are positioned at a first end of the chamber 16, which is elongate in a first direction. In the present exemplary embodiment, the chamber 16 is elongate in a horizontal direction and the inlet 28 and outlet 30 are positioned at a left-hand side of the chamber 16. The chamber 16 further includes tubing 32 which connects the inlet 28 and outlet 30 and defines a channel therebetween. The elongate shape of the chamber 16 allows for longer tubing 32. The tubing 32 is illustrated to be constructed from two pipes 32a connected by a drain 32b, this is to increase the length of the channel between the chamber inlet 28 and the chamber outlet 30. Optionally, the tubing 32 may extend in a helical manner from the inlet 28 to the insulated chamber 16 to the outlet 30 to increase the flow path between the inlet 28 and outlet 30. Other methods may be known to those skilled in the art and the present invention is not necessarily intended to be limited in this regard.

The tubing 32 is constructed from a thermally conductive material. Cooling modules 34 are positioned adjacent the outer surface of the tubing 32, and are in thermal contact with the tubing 32. In the present exemplary embodiment the cooling modules 34 are Peltier modules, also known as thermo-electrical cooling modules (TEC modules). Peltier modules are known in the art and therefore will not be described in further detail for conciseness.

It will be apparent to those skilled in the art that the Peltier modules 34 require a power source. Optionally, batteries 35 are supplied to provide such power. In order to provide enough power for adequate cooling of the Peltier modules, Lithium-ion batteries may be provided. Other suitable means of power supply, for example domestic power supply (also called mains, mains power or mains electricity), may be used and the present invention is not necessarily intended to be limited in this regard.

In FIG. 1 of the drawings, three Peltier modules 34 are illustrated however it will be apparent to those skilled in the art that the number of cooling modules 34 can be adjusted. The cooling ceramic plate of each Peltier module 34 is located adjacent the surface of the tubing 32. Heat sinks 36 are connected to the heating ceramic plate of each Peltier module 36 and configured to transfer thermal energy from the Peltier modules 34 to the outside of the thermally insulated chamber 16. Thus, the heat sinks 36 are constructed from thermally conductive plates 36a having a plurality of thermally conductive fins 36b extending perpendicularly from one side of the thermally conductive plate 36a. The fins 36b extend through the walls 17 of the insulated chamber 16 so as to direct heat away from the inside of the chamber 16. The fins 36b terminate in the cavity 15 between the walls of the insulated chamber 16 and the housing 10.

As described above, the cavity 15 is in fluid connection with the chamber inlet 28, and also with the split inlet 12 of the housing. Two separate flow paths are therefore defined, each configured to receive approximately 50% of the fluid input to the device. In other words, the input fluid can be split substantially evenly between the two flow paths. More specifically, in a first fluid flow path (shown in FIG. 1 to be on the right-hand side of the inlet 12) fluid (preferably water) can enter the cavity 15, flow over the fins 36b around the outside of the chamber 16, then enter the chamber 16. Where the fluid is water from the shower hose 20 (i.e. mains cold water), the water is cool enough to remove thermal energy from the fins 36b as it passes over them. When the water enters the chamber 16 it is cooled by the Peltier modules 34. In a second fluid flow path (shown in FIG. 1 to be on the left-hand side of the inlet 12), the fluid enters the cavity 15 via the inlet 12, then subsequently enters the chamber 16 via the chamber inlet 28. The first and second fluid flow paths (indicated by arrows in FIG. 1) therefore merge into a single fluid flow path on entry to the chamber 16.

Once the fluid is in the chamber 16, it flows through the tubing 32. The Peltier modules 34 cool the fluid to a desired temperature by drawing thermals energy away from the fluid. For some applications this temperature may be anything between −5° C. and 2° C. In order to prevent freezing and thus causing blockages in the cold therapy device, a preferred temperature of 0.5° C. is achieved. This is sufficient to administer cold therapy treatment and obtain the required results from cold therapy. It will be apparent to those skilled in the art that longer tubing 32 means more cooling can take place. Thus, the length of tubing can be adjusted in order to achieve the temperature required.

The now cooled fluid exits the insulate chamber via the chamber outlet 30 which feeds directly to a reservoir 38 to enable the cooled fluid to be exit the cold therapy device via the housing outlet 14 through all of the plurality of openings, thus creating the plurality of streams as described above.

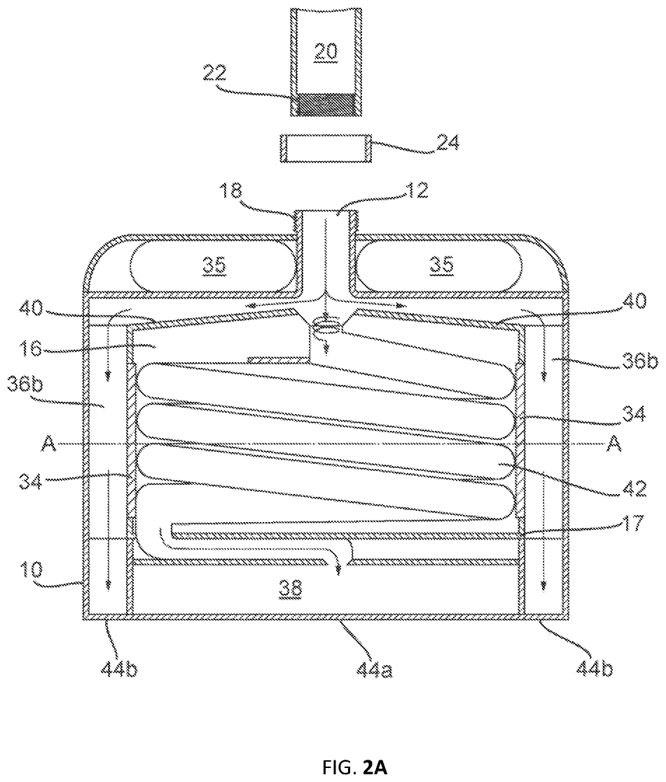

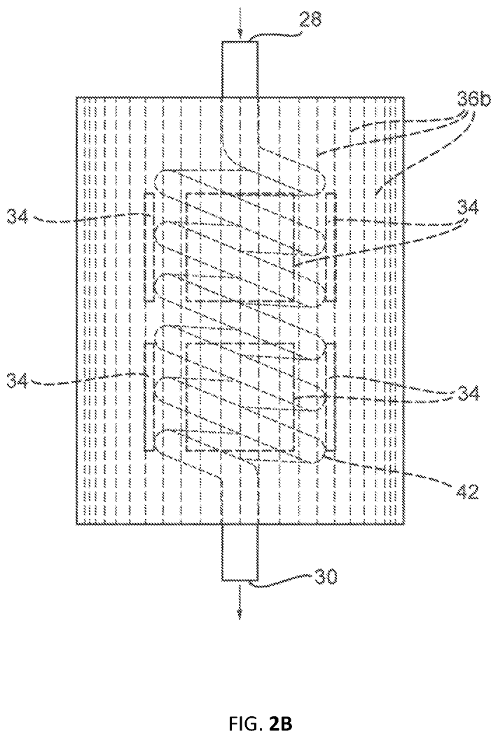

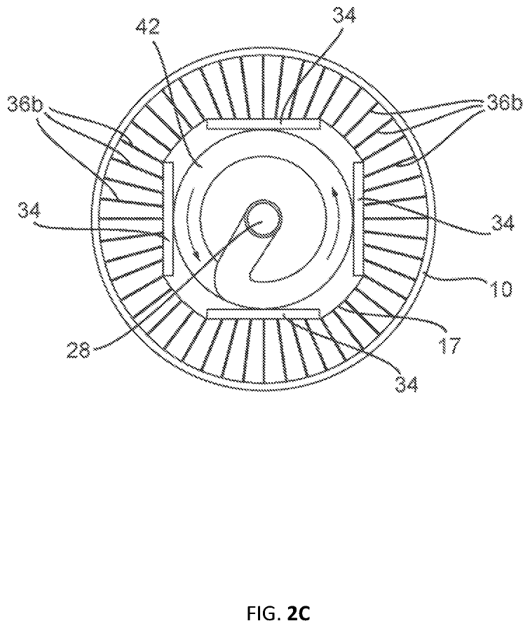

In a second embodiment of the invention, and referring to FIGS. 2A, 2B and 2C of the drawings, the cold therapy device is configured to split a single fluid flow path into two fluid flow paths after entry to the cavity 15 via the inlet 12. As in the first embodiment, the housing 10 defines a cavity 15 and includes an insulated chamber 16 having a chamber inlet 28 and a chamber outlet 30. The housing 10 and the chamber 16 are generally cylindrical, and in this embodiment the cavity 15 is an annular cavity 15 around the chamber 16. In this embodiment, the chamber inlet 28 is positioned substantially centrally, so that it is substantially aligned with, but spaced apart from, the housing inlet 12. The upper surface of the chamber 16, or roof 40, of the chamber 16 surrounding the chamber inlet 28 is pitched so that, when the cold therapy device is oriented for use on attachment to a shower hose 20, for example, any water which does not fall directly on the chamber inlet 16 flows away from the inlet 16 toward the annular cavity 15.

The chamber walls 17 have Peltier modules 34 tangentially contacting the walls. Four Peltier modules 34 are illustrated in FIG. 2B, however it will be apparent to those skilled in the art that additional modules 34 can be provided around the whole of the chamber 16. As before, the Peltier modules 34 may be powered by batteries 35. A single heat sink in the form of a plurality of fins 36b extending at right angles to the insulating walls 17 of the chamber 16 draw heat away from the modules 34 and aid cooling of the chamber 16. The fins extend into the cavity 15.

The tubing 42 is helical- or spiral-shaped and defines a flow path from the chamber inlet 28 to the chamber outlet 30. The chamber outlet 30, as with the previous embodiment illustrated in FIG. 1 of the drawings, connects directly with a reservoir 38, from which the fluid then exits the cold therapy device via the housing outlet 44. The housing outlet 44 consists of two parts. The first part, the central outlet portion 44a has the same diameter as the reservoir 38 and forms the lower surface of the reservoir 38, when the device is oriented for use. The second part, the peripheral outlet portion 44b encircles the central outlet portion 44a and forms the lower surface of the cavity 15, when the device is oriented for use. Both the central and the peripheral outlet portions 44a, 44b, include a plurality of small openings, for example 4 mm in diameter, configured to split the fluid stream into a plurality of streams for use as a shower. Additionally, the outlet 44 may include a misting device configured to diffuse water droplets in such a manner so as to deliver a fine mist to the user, in use.

In use, the cold therapy device is fitted to a domestic water source, for example a shower hose 20, the shower hose 20 in turn being connected to a domestic water supply and selectively controllable so that water can be selected to flow or not flow as required. The water enters the cavity 15 by the housing inlet 12 and follows one of two flow paths (illustrated by the arrows). As described above, because the chamber inlet 28 is positioned generally centrally (i.e. below the housing inlet 12, when the device is oriented for use) the majority of the water travels into the chamber inlet 28 and is cooled by the respective Peltier modules 34 as it flows through the tubing 42 into the reservoir 38. This defines the first fluid flow path. The proportion of fluid which uses the first fluid flow path may be substantially 70% of the total flow of water entering the cavity 15 from the shower hose 20. The cooled fluid is then split into a plurality of streams by the central outlet portion 44a.

In a second fluid flow path, a smaller portion of the total flow of water lands on the roof 40 of the chamber 16 and travels down the outside of the chamber walls 17. This portion of the fluid flow removes thermal energy from the heat sink fins 36 as it passes by them, aiding the cooling process. The smaller portion of fluid may be substantially 30% of the total flow of water entering the cavity 15 from the shower hose 20. Water which has travelled through the cold therapy device by the second fluid flow path is not cooled by the cold therapy device, but acts as a coolant for the heat sink and cooling modules 34. This fluid exits the device via the peripheral outlet portion 44b.

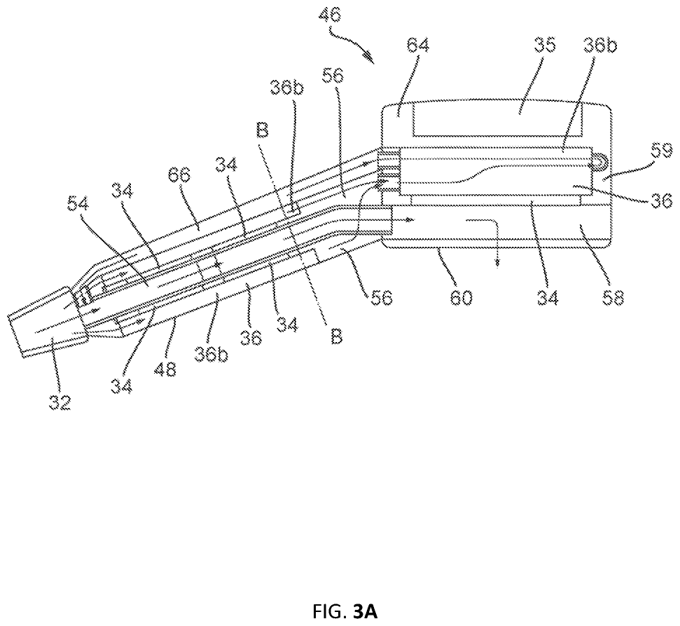

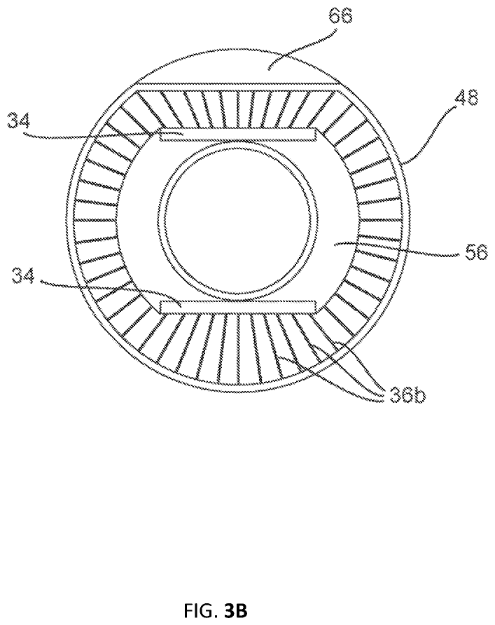

Referring to FIGS. 3A and 3B of the drawings, a cold therapy device according to a further embodiment of the invention is illustration, in the form of a hand-held shower head cold therapy device 46. The device 46 comprises a handle 48 fitted with a shower head portion 50 at one end of the handle 48. An attachment collar 52 is located on the opposing end of the handle 48 to the shower head 50. The collar 52 is configured to allow removable attachment of the device to a shower hose, in use. It will be apparent to those skilled in the art that the device 46 may be fitted to any suitable mains water conduit and the invention is not limited in this regard.

Referring in particular to FIG. 3B, which illustrated a cross-section of the handle 48, a primary cooling chamber 54 consisting of a linear channel runs substantially centrally along a longitudinal length within the handle 48 and allows water to flow from a first end of the handle 48 at the attachment collar 52, to the opposing end of the handle 48 at the shower head 50, when in use. Cooling devices in the form of Peltier modules 34 are in thermal contact with the walls of the primary cooling chamber 54, and are configured to cool the contents of the primary cooling chamber 54. Fleat sinks 36, comprising a base plate 36a and fins 36b are fitted to the Peltier modules in order to aid cooling. The fins 36b extend into an annular cavity 56 around the cooling chamber 54 within the handle 48.

The opposite end of the primary cooling chamber 54 is in fluid connection with a secondary cooling chamber 58 which is located in the lower portion of the shower head 50, when the device is oriented for use. The secondary cooling chamber 58 comprises an outlet 60 to allow water cooled to cold therapy temperatures to exit the device, for use. The outlet 60 may comprise a plurality of small apertures as would be familiar in a shower head. Additionally, the outlet 60 may include a misting device configured to diffuse water droplets in such a manner so as to deliver a fine mist to the user, in use.

Additional cooling modules in the form of Peltier modules 34 are in thermal contact with the secondary cooling chamber 58 and are configured to reduce the temperature of the contents of the secondary cooling chamber 58. The additional Peltier modules 34 have heat sinks 36 fitted thereon, which have fins 36 extending into an upper cavity 59. The upper cavity 59 is in fluid connection with the annular cavity 56. A cooling channel 66 is in fluid flow connection with the primary cooling chamber 52 and the upper cavity 59.

The device 46 is powered by batteries 35 which are located in a battery unit 64 at the uppermost portion of the shower head 50 and can be opened up for access in order to replace batteries as required.

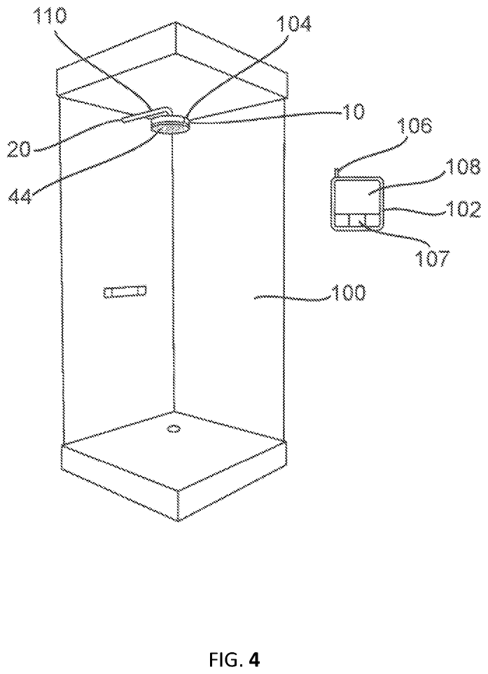

In use, when the device is connected to a mains water conduit, water first enters the handle 48 whereupon it splits into two paths. The first fluid flow path travels through the primary cooling chamber 54 into the secondary cooling chamber 58 and exits the device via outlet 60, whereupon a user may administer cold therapy treatment as required. In the second fluid flow path, water travels through the annular cavity 46, absorbing thermal energy from the fins 36b as it does, entering the upper cavity 59 of the shower head and absorbing thermal energy from the fins 36b located therein as it flows around the upper cavity 59. The water, having absorbed thermal energy and aiding the cooling of the primary and secondary cooling chambers 54, 58, exits the upper cavity 59 via the cooling channel 66 whereupon the water enters the primary cooling chamber 54 and joins with the first fluid flow path. Referring now to FIG. 4 of the drawings, a cold therapy system installed in a domestic shower 100 is illustrated. The cold therapy device of any of FIGS. 1 to 3B as described above may be used. The cold therapy device is installed at the open end of the shower hose 20 and water from a domestic water supply enters the cold therapy device and is cooled to cold therapeutic temperatures. An external control device 102 is provided. The control device 102 includes a transceiver 106, input means 107 for user input and a display 108. The transceiver is configured to communicate with a transceiver 102 in the housing 10 via any suitable means, for example by using radio. The control device 102 is operable by a user to alter the temperature of the cold water by signalling to the housing to reduce or increase power supplied to the Peltier modules 34. Thus the user advantageously have better control of the water temperature. In this embodiment, the cooling modules 34 are powered by mains electricity which is connected by a wire 110. This system is partially integrated and uses mains water and electricity. It can be retro-fitted to existing showers.

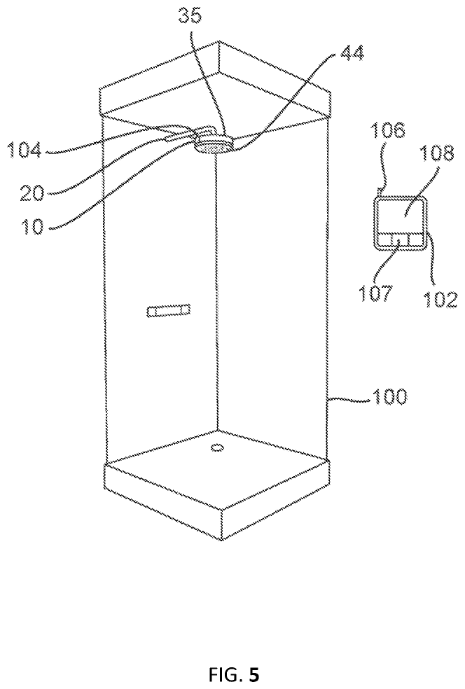

Referring to FIG. 5 of the drawings, a cold therapy system is illustrated in accordance with a second embodiment, including a cold therapy device as described in relation to any of FIGS. 1 to 3B of the drawings, a control module 102 communicably coupled to the cold therapy device via transceivers 104, 106 and having input means 107 and a display 108 for allowing a user to control the temperature of the cold water leaving the cold therapy device, wherein the Peltier modules are powered by battery or batteries 35 within the cold therapy device itself. This system is partially integrated and uses mains water. It can be retro-fitted to existing showers.

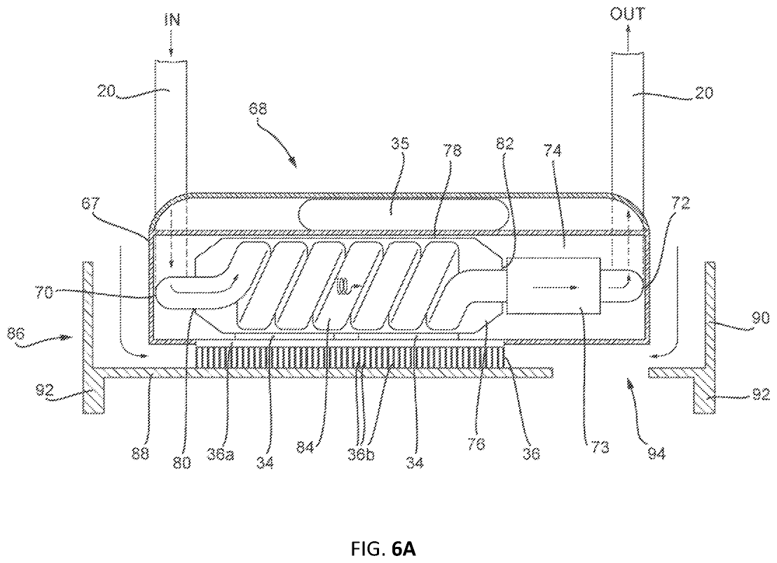

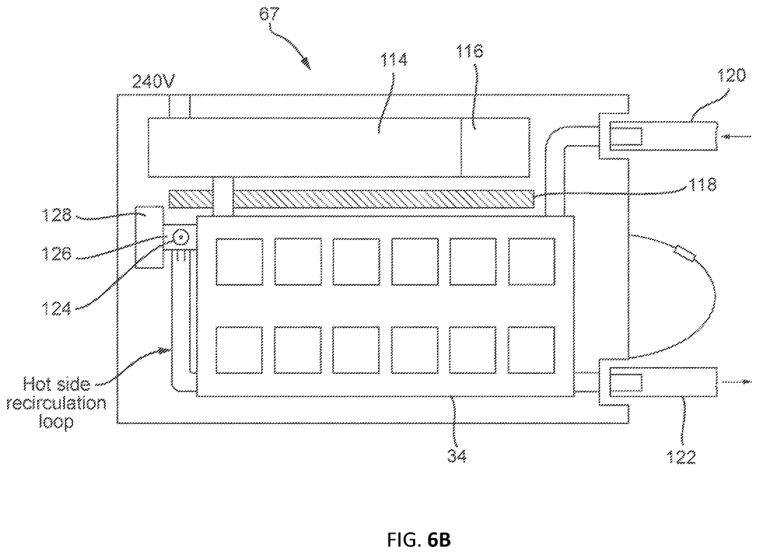

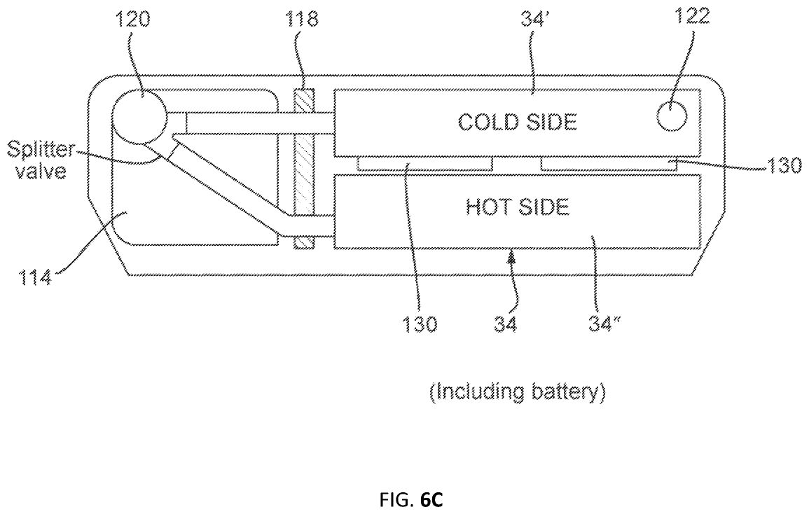

Referring to FIG. 6A of the drawings, a cold therapy device is illustrated. The cold therapy device comprises a cooling unit 68 in a housing 67 having an inlet 70 and an outlet 72 on a rear surface thereof. A first shower hose 20 can be connected to the inlet by, for example, a screw threaded portion (not shown). A second shower hose 20 can be connected to the outlet 72 by, for example, a screw threaded portion (not shown). A section of the first and second parts of the shower hose 20 are shown in dashed lines in FIG. 2A to illustrate where they continue behind the cooling unit 68. The waterproof housing 67 defines a cavity 74 having a thermally insulated chamber 76. The chamber 76 has insulating walls 78 which allow the temperature inside the chamber 76 to be much colder than the temperature outside the chamber 76. The chamber 76 has a chamber inlet 80 and a chamber outlet 82. Tubing 84 extends from the inlet 70 to the housing 67, through the chamber 76 via the chamber inlet 80 to the outlet 72 to the housing 67, via the chamber outlet 82. Thus the tubing defines a single fluid flow path from the inlet 70 to the outlet 72 of the housing 67.

A pump 73 is provided along the tubing 84 at a point between the outlet 82 of the chamber 76 and the outlet 54 of the housing 67. The pump 73 is configured to aid in flow of fluid in the direction toward the outlet 72 of the housing 67, and upwardly through the second part of the shower hose 20, in use.

The tubing 84 forms a spiral, or helical, shape as it extends through the chamber 76. Cooling modules 34, in this case Peltier modules though other cooling modules may be known to those skilled in the art, are in thermal contact with the chamber 76. In FIG. 3A of the drawings, two Peltier modules 34 are illustrated, though it will be apparent to those skilled in the art that any number of modules 34 may be used. The cooling modules 34 cool the internal temperature of the chamber 76. The helical tubing 84 provides a longer fluid flow path through the chamber 76 thus providing more time for the fluid therein to be cooled. As with previous embodiments, the Peltier modules 34 may be powered by one or more batteries 35.

A heat sink 36 is attached to the Peltier modules 34 on the hot ceramic plate of the modules 34. As with previous embodiments the heat sink includes a thermally conductive plate 36a and a plurality of thermally conductive fins 36b extending substantially perpendicularly from the plate 36a. The fins 36b extend to a space external to the housing 67 of the cooling unit 68. The plate 36a forms part of the lower side of the waterproof housing 67, and any joins between the housing and the plate 36a are sealed for both waterproofing purposes and thermal insulation purposes.

A collection tray 86 is shaped and configured to be large enough to receive the cooling unit 68 to be placed therein, as illustrated. The tray 86 includes a base 88, at least one wall 90 around the perimeter of the base 88 and extending perpendicularly thereto in one direction, and feet 92 positioned around the base 88 extending in an opposing direction to the wall 90. The wall 90 may be a single continuous wall (for example, in the case of the tray being generally circular or oval in shape), or may optionally include a number of walls at an angle to each other (in the case of the tray being generally polygonal in shape). The number of feet 92 is such that the base 88 can be substantially stable in use when placed on a wet surface, and the exact configuration of the feet is not intended to be limited in the present invention. The tray 86 further comprises a drain 94 through the base 88. The drain 94 is located to one side of the base 88 to allow fluid to flow along the base 88 before exiting the tray 86.

In use, water from the shower hose 20, which in turn comes from a domestic water supply, follows the fluid flow path as indicated by the arrows into the cooling unit 68, through the tubing 84 from the inlet 70 toward the outlet 72. The water is cooled to cold therapeutic temperatures by the Peltier modules as it travels through the helical tubing 84 in the chamber 76. The pump 73 enables the cold therapeutic water to exit the cooling unit 68 and pushes it upwardly through the second part of the shower hose 20. From there, the cold therapeutic water may exit the shower hose through a shower head, for example (not shown), whereupon it can be used for administering cold therapy. The runoff is collected in the collection tray 86 where it flows along the base 88 via the heat sink fins 36b, before draining away via the drain 94. Thus the runoff can be used to improve cooling of the fluid going through the cooling unit 68, by drawing thermal energy away from the heat sink fins.

Referring to FIG. 6B of the drawings, a portable cold therapy device according to an exemplary embodiment of the present invention is illustrated. The device comprises a cooling module or heat exchanger 34in a housing 67. A transformer 114 and central processing unit (CPU) 116 are provided within the housing 67, with an insulating layer 118 located between the heat exchanger 34 and the transformer/CPU 114/116.

The transformer 114 may be electrically connectable to a power supply (e.g. 240V electrical power supply), and is electrically coupled to the heat exchanger, to provide power thereto when the device is switched on via the CPU 116. A cold water inlet 120 is provided at one end of the heat exchanger 34, extending into the housing 67 on one side. Water entering the heat exchanger 34 via the inlet 120 is cooled to cold therapy temperatures, as before, and exits the heat exchanger 34 (and housing) 67 via a cold water outlet 122. A diverter valve 124 may be provided at the ‘hot’ side of the heat exchanger 34, at a second outlet 126, such that excess warm water can either be fed back (or recirculated) to the heat exchanger 34 for cooling, or fed to a plug outlet 128 for draining away as waste. As shown in FIG. 6C of the drawings, Peltier modules 130 may once again be used in the heat exchanger 34 to provide a ‘cold side’ 34′ and a ‘hot side’ 34″ within the heat exchanger 34, as before. A splitter valve 132 is provided at the inlet 120 such that the cold water entering the unit can be controlled.

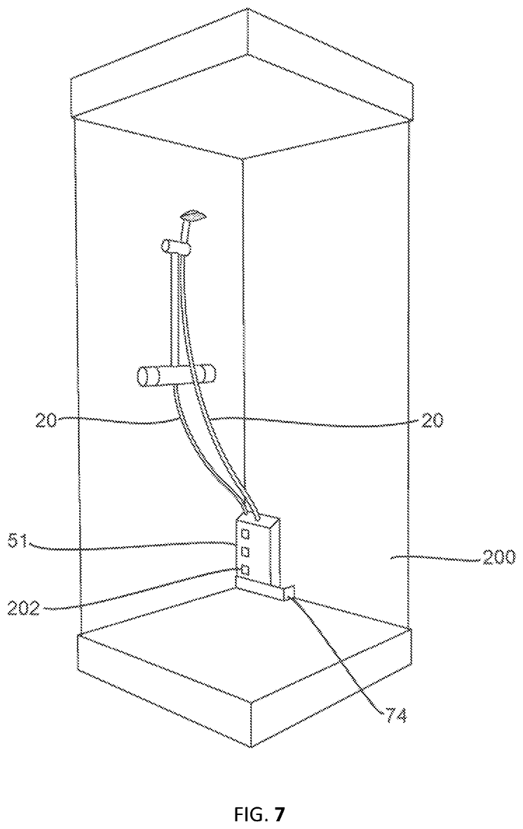

Referring to FIG. 7 of the drawings, a cold therapy system in accordance with an exemplary embodiment of the invention is illustrated, including a shower 200, having a cold therapy device of FIG. 6A fitted onto the shower hose 20 and placed. The cooling unit 51 of the cold therapy device includes input means 202 for altering the power supplied to the Peltier modules, therefore allowing adjustability of the cold-therapeutic water temperature. The input means 202 may be a dial, buttons, or other input means as will be known in the art. This system can be installed and removed from any existing shower system, and can be portable.

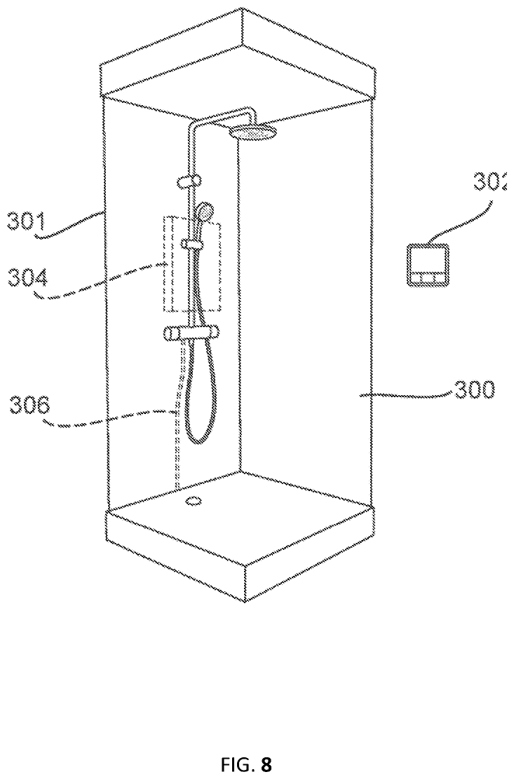

Referring to FIG. 8 of the drawings, a cold therapy system is illustrated, including a shower 300 having the cold therapy device of FIG. 6A installed in the shower unit 304 itself. In this embodiment, the cooling unit 51 is within the shower unit 304, and the tray 74 is replaced by a drainage conduit 306. The drainage conduit 306 is provided to drain away water which has been used to cool the heat sink 36 of the cold therapy device. The cold therapy device is still fitted to the shower hose 20 however it is not visible as a separate device as the cooling unit 51 of the cold therapy device is integrated with the shower unit 304. The shower unit 304 is a conventional shower unit as will be known in the art. The shower unit 304 is installed behind the wall 301 of the shower 300. The system optionally further includes a control device 302 configured to communicate with the cold therapy device within the shower unit 304 via, for example, radio via transceivers, to provide adjustability of the temperature as described with previous embodiments. This system is fully integrated, uses the domestic water supply and electricity, and would be installed on installation of the shower 300.

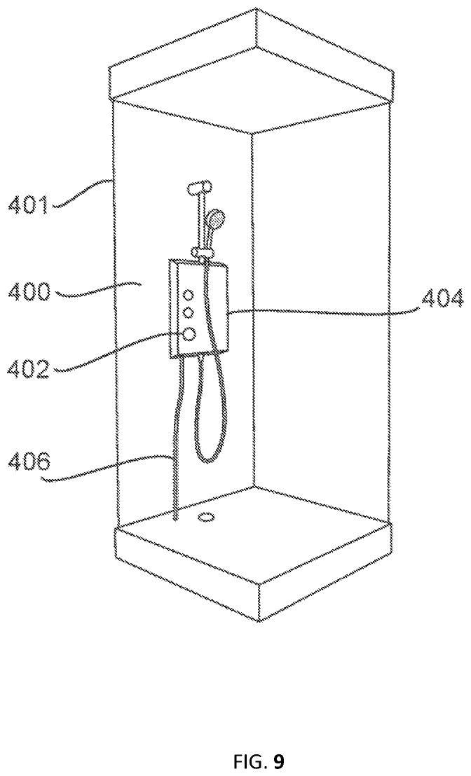

Referring to FIG. 9 of the drawings, a cold therapy system is illustrated including a shower 400 having a shower wall 401, a shower unit 404 having input means 402 thereon and a cooling unit 51 of the cold therapy device of FIG. 5 integrated therein, and a waste conduit 406 (replacing the tray 74 of the cold therapy device of FIG. 5) for draining away water used to cool the heat sink 36 of the cold therapy device within the shower unit 404. In this system, the shower unit 404 is installed on the outside of the shower wall 401 and the input means 402 allow the user to adjust the temperature as required. This system is partially integrated, uses the domestic water supply, and can be installed on existing showers.

The above embodiments have been described with reference to a shower system, however it will be apparent to those skilled in the art that the system can be used with a faucet, or other domestic water delivery device connected to a domestic water supply known in the art.

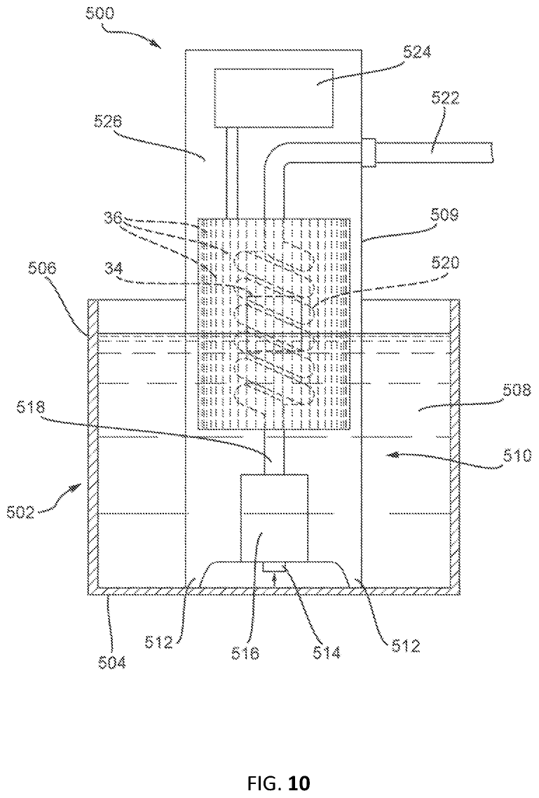

Referring now to FIG. 10 of the drawings, a portable cold therapy device according to an embodiment of the invention is illustrated comprising a cooling chamber 500 and a refillable reservoir 502. The reservoir 502 comprises a waterproof bucket having a base 504 and at least one wall 506 extending perpendicularly to the base 504. The reservoir 502 may be circular in shape (thus requiring one, single continuous curved wall 506) or polygonal (thus requiring a plurality of walls 506 joined together). The reservoir 502 is configured to receive the cooling chamber 500 and an amount of water 508, such that the water at least covers the lower half of the cooling chamber 500.

The cooling chamber 500 has a cylindrical housing 509 with a flattened roof and base. When oriented for use the lower end 510 of the housing 509 has a plurality of feet 512 extending from an edge of the base of the cooling chamber 500, so as to lift the cooling chamber 500 base away from the base 504 of the reservoir 502. An inlet port 514 located generally centrally on the base is configured to allow water 508 therethrough into the cooling chamber 500. The inlet port 514 is connected to a pump 516 which is configured to draw water 508 from the reservoir 502 into the cooling chamber 509.

Tubing 518 extends from the pump 516 to a helical tubing portion 520 which has cooling modules 34 in the form of Peltier modules 34 in thermal contact therewith. Fleat sinks 36 are in thermal contact with the cooling modules 34 and extend into an annular cavity 526 within the cooling chamber 500 but having no water flow therethrough. Water 508 is pumped through the helical tubing portion 520 by the pump 516 to a delivery conduit 522 configured to deliver cooled water for cold therapy use. A power unit 524 is located at the upper end of the cooling chamber 500 and may include a power source, for example a battery, configured to power the pump 516 and cooling modules 34.

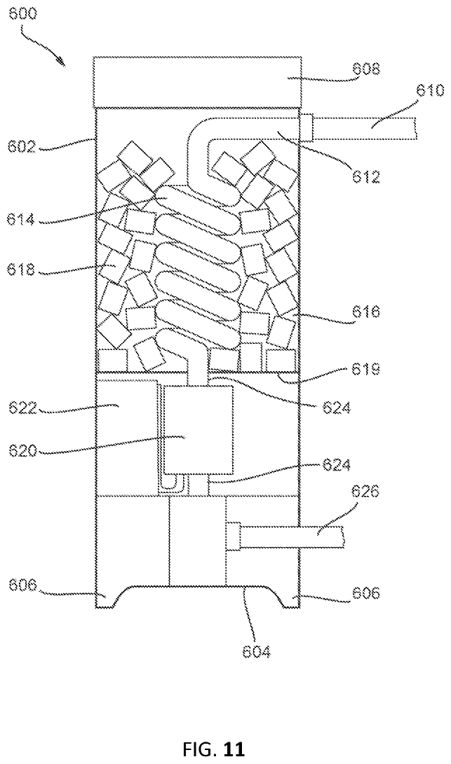

Alternatively, and with reference to FIG. 11, the cold therapy device may comprise only a cooling chamber 600 having cylindrical housing 602 with a base 604 having a plurality of feet 606 extending from the edge of the housing 602 so as to lift the base 604 away from the surface it is placed on, in use. At an opposing upper end of the housing 602 the housing is open. A removable lid 608 is provided to fit over the open upper end.

The water inlet 610 is located on the side wall of the housing 602 near an upper edge. Tubing 612 extends between the inlet 610 and a helical tubing portion 614. The annular cavity 616 around the helical tubing portion 614 can be accessed via the open upper end of the housing 602 to allow a user to deposit, for example, ice 618 in order to aid cooling of the contents of the helical tubing portion 614.

The lower end of the housing is separated from the upper end by a divider plate 619. The divider plate is configured to waterproof the lower end of the housing 602. A pump 618 located in the lower end of the housing is provided to draw water through the helical tubing portion 614 from the inlet 610. The pump 620 is powered by a power unit 622 which may contain, for example, batteries. Tubing 624 extends from the end of the helical tubing portion to a delivery conduit 626 configured to deliver cooled water for cold therapy use.

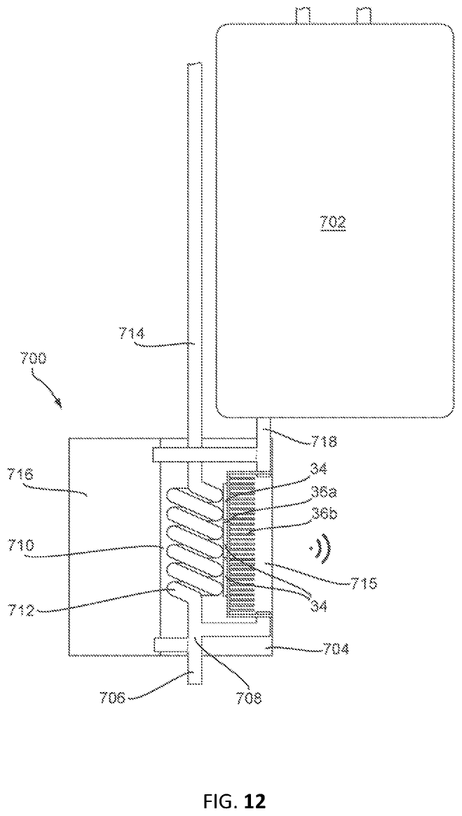

In yet another embodiment, and with reference to FIG. 12 of the drawings, there is provided a cold therapy system comprising a cooling device 700 fitted to a boiler system 702.

The cooling device 700 comprises housing 704 having an inlet 706 for receiving water from a domestic water supply. The inlet 706 is connected to tubing 708 which directs water primarily into cooling chamber 710. As with previously described embodiments, the cooling chamber 710 comprises helical, or spiral, tubing 712. Cooling modules 34 are in thermal contact with the helical tubing 712 and configured to draw thermal energy away, thus cooling the contents of the helical tubing 712. Heat sinks 36 are fitted to the cooling modules 34, and comprise a base plate 36a having a plurality of fins 36b extending substantially perpendicularly thereto. In FIG. 12, three cooling modules 34, in the form of Peltier modules 34, are illustrated with a single heat sink 36 spanning the entire three Peltier modules, however it will be apparent to the person skilled in the art that the number of cooling modules 34 and heat sinks 36 can be changed as required, and the invention is not necessarily intended to be limited in this regard. Heat sink fins extend external to the cooling chamber 710 into a cavity 715. Cooled water exits the cooling chamber 710 via a delivery conduit 714 for use in cold therapy.

Before the water reaches the cooling chamber 710, an offshoot tube 714 directs some of the water into the cavity 715 so that the water passes over heat sink fins, in order to aid cooling. A storage tank 716 in fluid connection with the cavity 715 stores water which has been used to cool the heat sink 36 when the boiler is not in use. A waste water conduit 718 allows water to flow from the storage tank 716 and the cavity 715 into the boiler when the boiler is in use. This ensures no water is wasted during use. This also allows water to flow from the mains water supply or other domestic water supply directly into the boiler without going through the cooling chamber first for unnecessary cooling, when the boiler is required to be used.

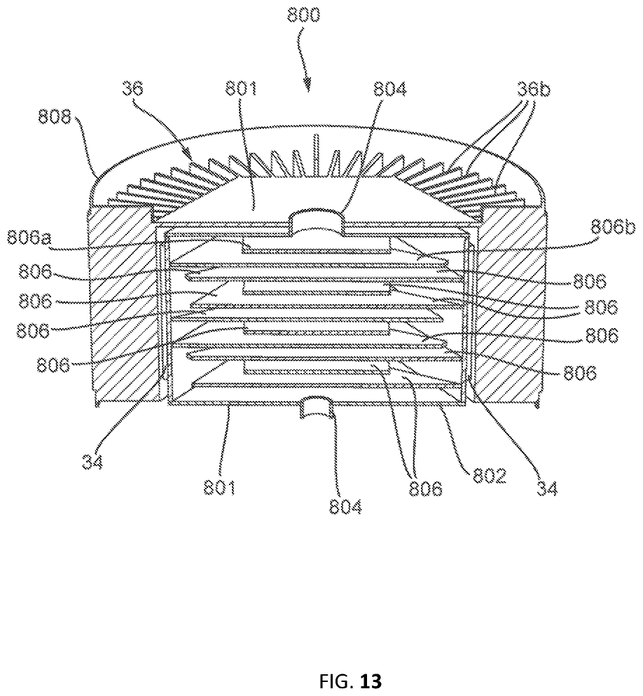

Referring to FIG. 13 of the drawings, an alternative cooling chamber 800 is illustrated. The chamber 800 can be used with any of the above described embodiments which require a cooling chamber 800. The chamber 800 comprises a sealed chamber 802 having a hexagonal top and base 801 joined by six side walls. The top and base 801 each have an aperture 804 located therethrough located generally centrally which allows water flow. The apertures 804 may be an inlet or an outlet depending on direction of water flow. A plurality of plates 806 are located in the chamber 802 and extend from a side wall of the chamber 802 in a horizontal direction toward the diametrically opposing wall. Eleven plates 806 are illustrated in FIG. 13 however it will be apparent to those skilled in the art that any number of plates 806 may be used depending on the requirements of the cooling chamber.

Each plate 806 only extends part of the total distance between opposing walls thus creating a gap between the opposing wall and the end of the plate. The first plate 806a is located at the top of the chamber and extends from a first side wall.

The next plate 806b extends from a wall adjacent the wall the first plate 806a was extending towards. Each subsequent plate 806 extends in this way so that gaps between the ends of each plate 806 and the side wall each plate 806 is extending toward are shifted by 120° and alternate sides of the sealed chamber 802. Thus the water flow is forced to take a longer path through the chamber 802. Cooling modules 34 in the form of Peltier modules 34 are located on each side wall outside the sealed chamber 802, and heat sinks 36 are fitted around the Peltier modules 34 to aid cooling. Housing 808 encloses the whole cooling chamber 800.

In embodiments including a remote control device to control the water temperature, it will be apparent to the person skilled in the art that sequences may be programmed so that delivery of water at certain temperatures is provided to the user. For example, an app on a mobile device may include means by which the use can select a pre-set sequence. The app then transmits this selection to the cold therapy device or system via transmitters which then alters the power provided to the cooling modules in accordance with the instructions provided. Thus, the control device (in this example a user mobile device) can be used to deliver a sequence of cold therapy. In any event, a CPU may be provided or associated with the system as described in relation to one or more of the embodiments described above, which may be used to control a series of valves directing water through heat exchangers and the power to the different Peltier modules to deliver different cold therapy effects.

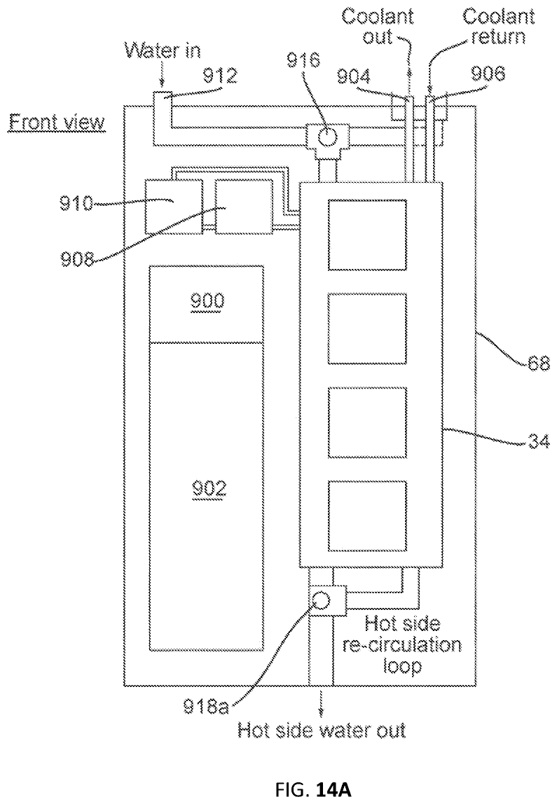

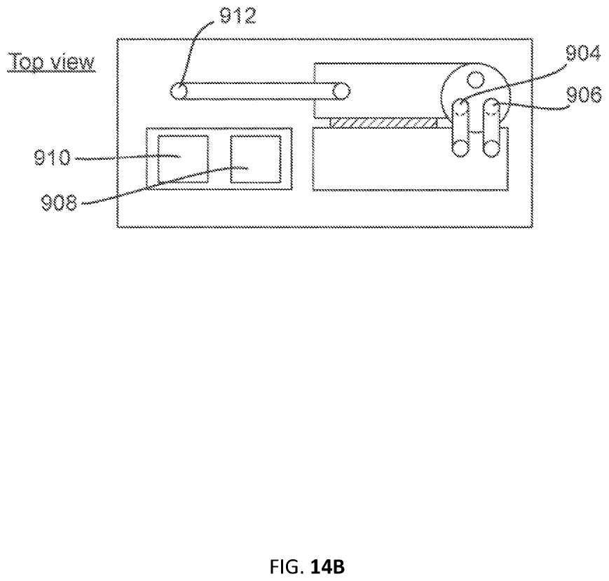

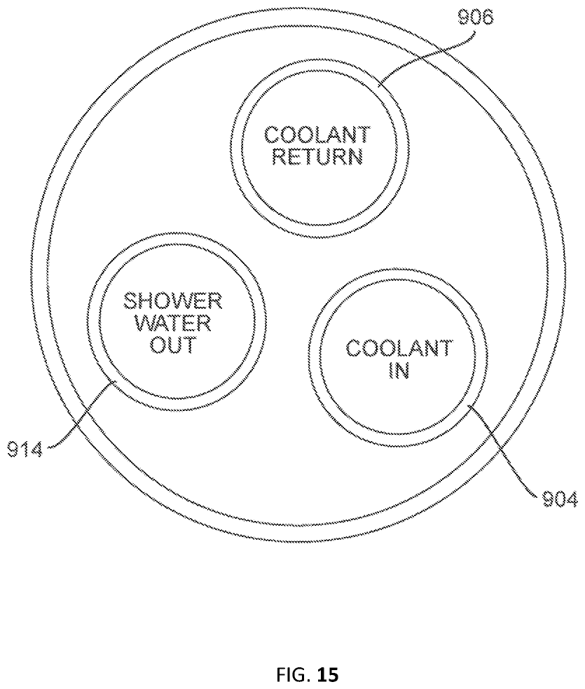

Referring to FIGS. 14A, 14B and 15 of the drawings, a cold therapy device according to an exemplary embodiment of the invention, comprises a CPU and power supply 900, 902 in a housing 68. In this case, the heat exchanger 67 is coupled to a reservoir 908 filled with a pre-cooled coolant fluid, such as glycol. Alternatively, the pre-cooled coolant fluid may be any suitable gas or liquid known in the art. When the unit is switched on, a pump 910 pumps the coolant into the heat exchanger 67 to substantially immediately start cooling the ‘cold side’. Cold water enters the heat exchanger 67 via an inlet 912 and is cooled by the heat exchanger 67 to cold therapy temperatures and output to a shower head (or other outlet) for delivery to a user via the shower outlet aperture 914. A splitter valve 916 is located at the inlet 912 for controlling the incoming cold water. Once again, a ‘hot side’ re-circulation loop 918 including a diverter valve 918a is provided at the ‘hot side’ of the heat exchanger 67. The shower head (or other delivery device) may include health and/or physiological sensors for monitoring health/physiological parameters or characteristics of a user and generating data representative thereof, for storage or display locally and/or transmittal to a remote processing facility for analysis and/or display (via an associated app, for example). One or more infra-red heaters may be provided in the shower head, which may or may not be configured to pulse, for providing directed heat to a part of the user during use of the system.

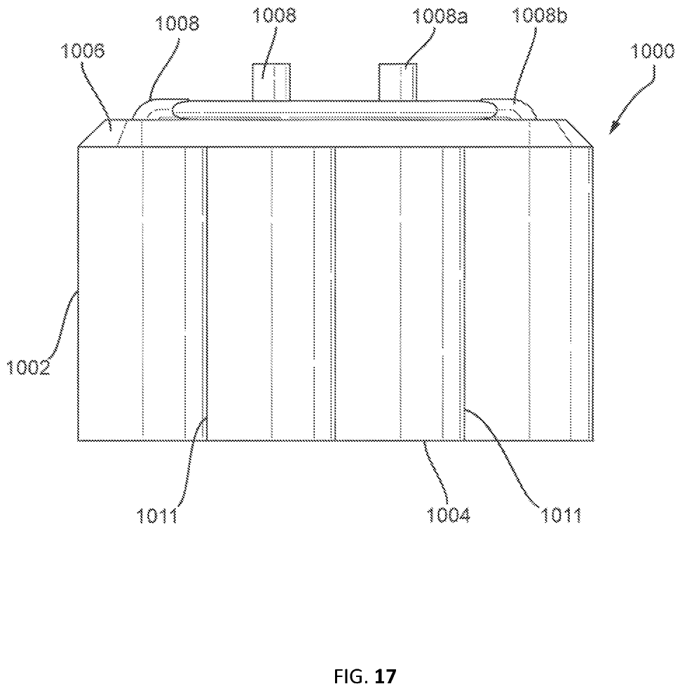

Referring to FIGS. 17, 18 and 19 of the drawings, there is illustrated a cold therapy device according to yet another embodiment of the invention.

The cold therapy device 1000 comprises a plurality of cooling chambers 1002. Each cooling chamber 1002 is generally cylindrical in shape and has a substantially flat closed circular base 1004. Base 1004 defines the bottom of the device when oriented for use. While the present embodiment is described as being generally cylindrical, other shapes may be use such as polygonal. At the upper end, each chamber 1002 terminates in a pitched roof 1006. Where multiple cooling chambers 1002 are arranged in parallel, as in FIG. 17, pitched roofs 1006 are connected via metal work. Thermally insulating walls extend between the roof 1006 and base 1004. Walls may be double walls having a vacuum or near vacuum therein. Walls may be double walls including a coolant fluid or gel such as glycol or water, or other suitable coolant known in the art.

For each chamber 1002, the pitched roof 1006 has a conduit 1008 affixed centrally, allowing fluid flow communication to the cooling cavity 1010 defined by the cooling chamber 1002. Depending on the direction of fluid flow, conduit 1008a is a water inlet configured to receive water to be cooled and 1008b is a water outlet configured to deliver cooled water. If the water is configured to flow the other way through the system, then conduit 1008a is a water outlet configured to deliver cooled water and conduit 1008b is a water inlet configured to receive water to be cooled. In alternative embodiments conduit 1008 may be split so that the water flow is divided on entry.

Near the base of each cooling chamber 1002 is a side flow conduit 1011 which enables two cooling chambers to have fluid flow communication between them, such that they are connected in series. Pairs of chambers 1002 may then be connected in parallel.

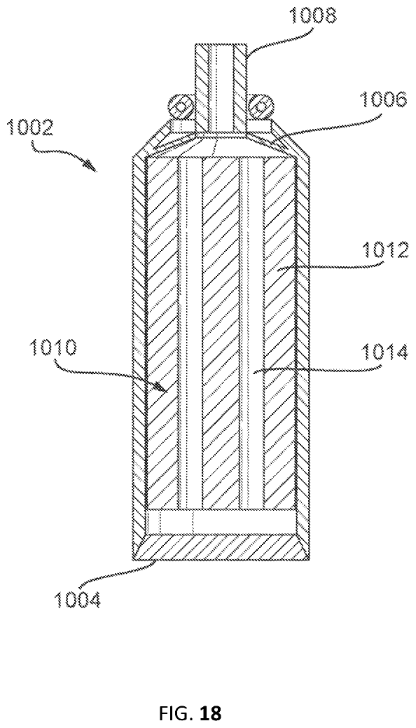

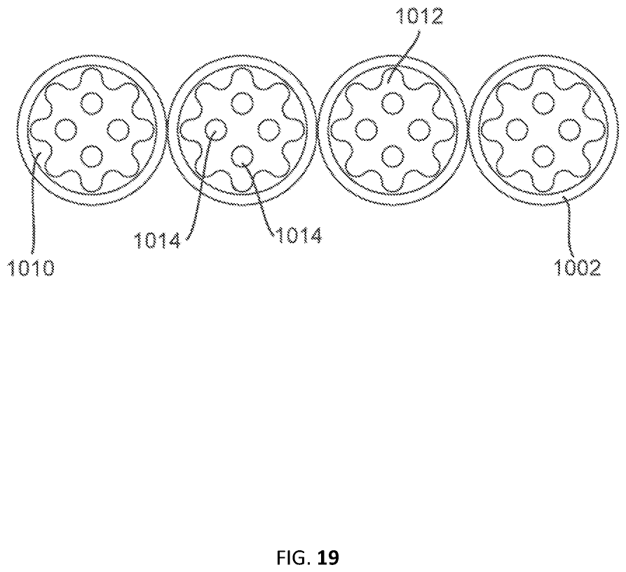

Referring to FIGS. 18 and 19 in particular, the cooling cavity, indicated generally at 1010, includes a shaped insert 1012 which divides the cavity 1010 and creates a plurality of channels 1014 therethrough. The insert may be hollowed and have coolant located therein. Coolant may be glycol or water, or any other suitable coolant known in the art. Insert 1012 therefore functions as a passive cooling module 34.

In use, inserts 1012 and chambers 1002 are first cooled via refrigeration means. For example, a user may place each component into their home freezer or refrigerator. When the components of the device have been manually cooled, the device 1000 is assembled as shown in FIG. 17 by simply connecting together each part to the relevant conduits. Water flows into conduit 1008a, which is configured as a water inlet, and is divided among the cooling channels 1014 within the cooling chamber cavity. When water reaches the base of the first cooling chamber 1002, it flows in a sideways fashion toward the second cooling chamber in the series. Due to the continuous flow of water at the conduit 1008a, water is driven up through cooling channels 1014 in the second cooling chamber 1002 until it is delivered at conduit 1008b ready to be used in cold therapy treatments.

For example, conduit 1008a, when configured as a water inlet, may be connected to a faucet or other domestic water supply. For example, conduit 1008b, when configured as a water outlet, may be connected to a shower head or may simply be used straight from the conduit 1008b mouth.

In total, in the present exemplary embodiment the combined surface area of the channels may be up to 60 m2. In use, water will flow through the device at substantially 8 litres per minute. Slower flowing water may require fewer channels as the amount of time spent in the channels is increased. Alternatively, faster flowing water may require more channels. For example, a flow rate of 6 litres per minute or less may be used. Optionally, a flow rate of up to 12 litres per minute may be used. Optionally, a flow rate of over 12 litres per minute may be used. In an exemplary embodiment the device may be configured to cool input water to the device by at least 5° C. Optionally, the device may be configured to cool input water to the device by at least 10° C.

The number of channels 1014 can be varied as required. If the ambient water table temperature of a locality is higher (e.g. Australia), then the number of channels can be increased. This in turn increases the surface area which comes into contact with the water for a greater cooling effect. In a locality where the ambient water table temperature is not as high (e.g. England), less channels are required to achieve the required cooling effect.

This enables water to be cooled to a colder temperature than would normally be achieved simply by turning on the cold tap. The cooled water is then applied to the body as required for therapeutic application (for example, sports injury, soothe inflammation, etc.). This provides the user with an at-home cold therapy device which is portable and can be attached and detached as required. Any of the above described embodiments may further include a temperature regulation circuit configured to prevent water temperature dropping so low that the water freezes and block the device or system. Such a temperature regulation circuit may, for example, include a timer configured to switch cooling modules 34 off after a set amount of time. Alternatively, a thermometer within the device may trigger the cooling modules 34 to turn off once a threshold temperature has been reached.

Thus the embodiments described above provide a user with a means for cooling water efficiently and effectively from a water supply, such as a water tank, reservoir, or mains water supply, to cold therapy temperatures, without requiring expensive equipment. Moreover, in embodiments of the invention, the cold therapy device and system is installable with current water delivery devices already known in the home, for example showers or baths. Advantageously, embodiments of the invention can be simply and cheaply installed in gyms, hotels, spas, offices, hospitals and other such sites. Embodiments of the invention are suitable for being installed in almost any commercial or domestic setting. Thus cold therapy can be more widely available. It will be apparent to those skilled in the art that variations and alternatives to the above-described embodiments not described above may still fall within the scope of the appended claims.

For example, the hand-held devices and system embodiments which may be used for specific targeting of particular areas in partial body cold therapy may further include infra-red heat lamps located in the outlet configured to deliver a warming sensation, in use. These infra-red lights or heat lamps may or may not be configured to pulse, in use. This further supplements and compliments the benefit of the cold therapy by providing a warming element.

Control means may be provided and configured to alter the water temperature rapidly by using both sides of the Peltier modules and a split heat exchanger to deliver rapidly changing water temperatures.

Loudspeakers may be integrated or otherwise provided in or with a system according to the invention so as to enable music or other media to be played.

Sensors may be provided in the system for monitoring body temperature changes through thermal imaging, for example, in order to identify ‘hot spots’ that may highlight health issues. Thus, thermal imaging cameras may be provided for this purpose. Indeed, other health and/or physiological monitoring sensors may be provided for collecting health/physiological data from a user, and means may be provided for storing and/or displaying the data locally and/or communicating such data to a remote processing facility for analysis and/or display (by means of, for example, an associated app). The thermal imaging device(s) and/or the health/physiological sensors could be configured to provide the ability to test users for fever, but also, in the case of a pandemic such as COVID-19, or a recurring/second pandemic wave, they can be used to an test users and supply doctors, hospitals, and places of work, for example, peace of mind that a user is not infected, as well as allowing a sporting professional to prove that they are fit enough to compete in an event. Such sensors may be short-range, non-contact sensors, such as an infra-red thermometer that monitors a user for fever. The sensors may be provided in, for example, a shower head included in a system according to the invention, and they may be configured to communicate wirelessly with a remote processing facility and/or wires may be provided that run from the shower head through the hose to allow transmittal of power and/or data as required.

A chilled cooling tank may be provided in between the water supply and the cooling devices of the above described embodiments. In this way the water input to the cold therapy device is already at a lower base temperature in order to improve cooling. The chilled cooling tank may cool input water by compression cooling or other refrigeration means.

1. A cold therapy device for use in a water delivery system which is coupled to a mains water supply, the device comprising:
a housing having an inlet for receiving water from a mains water supply, wherein the housing comprises at least one cooling module configured to reduce the temperature within the housing to cold therapeutic temperatures, so that, in use, when water is received therein from the inlet, it is cooled to cold therapeutic temperatures; and
an outlet for delivering water from the housing on to a user.

2. The cold therapy device according to claim 1, wherein the device is shaped as a shower head attachment.

3. The cold therapy device according to claim 1, wherein the outlet is a plate having a plurality of holes therethrough, so that water is delivered as a shower.

4. The cold therapy device according to claim 1, wherein the cooling module comprises an enclosure having at least one cooling member therein and means for receiving a coolant fluid within a region adjacent said cooling member.

5. The cold therapy device according to claim 4, wherein said enclosure includes an inlet for receiving said coolant fluid.

6. The cold therapy device according to claim 5, further comprising means for selectively cooling said coolant fluid and delivering cooled coolant fluid to said enclosure.

7. The cold therapy device according to claim 1, wherein the inlet is configured to be removably attached to a shower inlet hose, and the outlet is configured to be removably attached to a shower outlet hose.

8. The cold therapy device according to claim 1, wherein the housing comprises a chamber having a removable insert therein defining a plurality of channels for fluid flow.

9. The cold therapy device according to claim 8, wherein the removable insert is filled with a coolant fluid.

10. The cold therapy device according to claim 1, wherein the at least one cooling module is a Peltier module.

11. The cold therapy device according to claim 1, further comprising a thermally insulated chamber within the housing having an inlet in fluid communication with the housing inlet and an outlet in fluid communication with the housing outlet, wherein the at least one cooling module is adjacent to the chamber and configured to reduce the temperature within the chamber.

12. The cold therapy device according to claim 9, wherein the chamber comprises tubing defining a flow path from the chamber inlet to the chamber outlet.

13. The cold therapy device according to claim 1, wherein the at least one cooling module comprises a heat sink.

14. The cold therapy device according to claim 13, the housing comprising a cavity in thermal contact with the heat sink of the at least one cooling module.

15. The cold therapy device according to claim 14, wherein the cavity comprises an inlet for receiving water from a domestic water supply and an outlet for delivering water received in the cavity, so that, in use, thermal energy is drawn away from the heat sink.

16. The cold therapy device according to claim 1, comprising a selectively actuatable power source for delivering electrical power to the at least one cooling module.

17. The cold therapy device according to claim 1, configured to cool water to between 0.5° C. and 5.5° C.

18. The cold therapy device according to claim 1, configured to cool water input to the device by at least 5° C.

19. A cold therapy kit for use in a water delivery system which is connected to a water supply, comprising:
a cold therapy device according to claim 1 comprising attachments for fitting the cold therapy device to a domestic water delivery unit, the water delivery unit being configured to deliver water to the cold therapy device for cooling, and input means for selectively controlling the at least one cooling module.

20. A method of adapting a water delivery system to deliver water cooled to cold therapeutic temperatures, using a cold therapy device having an inlet for receiving water and an outlet for delivering cooled water, and having a cooling module therebetween, the method comprising the steps of:
identifying the fluid flow path between the water source and the point of delivery of water;
making a discontinuation in the fluid flow path to create a first flow path coming from the domestic water source to the discontinuation and a second flow path going from the discontinuation to the point of delivery of water;
attaching the inlet of a cold therapy device to the first flow path at the discontinuation; and
attaching the outlet of a cold therapy device to the second flow path at the discontinuation, so that the fluid flow path is restored and water flows through the cooling module of the cold therapy device.
